(19) United States
(12) Patent Application Publication
(10) Pub. No.: US 2007/0292687 A1
(43) Pub. Date: Dec. 20, 2007

(54) Microcapsule with Controllable or Delayed Release for the Immobilization of Chemical and/or Biological Materials, and Method for the Production Thereof

(76) Inventor: Rainer Pommersheim, Mainz (DE)

Correspondence Address: BODNER & O'ROURKE, LLP, 425 BROADHOLLOW ROAD, SUITE 108, MELVILLE, NY, 11747, US

(21) Appl. No.: 10/587,494

(22) PCT Filed: Jan. 31, 2005
(86) PCT No.: PCT/EP05/00927

§ 371 Date: Jul. 2, 2007

(30) Foreign Application Priority Data
Jan. 31, 2004 (DE) 10 2004 005 054.6
Feb. 23, 2004 (DE) 10 2004 00 051.3

(51) Int. Cl. A61K 9/16

(52) U.S. Cl. 428/402.2

(57) ABSTRACT

The invention relates to a microcapsule, especially for the immobilization of chemical and/or biological material, and to a method for the production thereof, which is stable in concentrated media, but is destroyed, upon diluting the medium, already under a relatively small mechanical stress, so that the encapsulated material is released. In accordance with the present invention, this material may be both a chemical substance, such as an active substance or an enzyme etc., or also a biological material, such as microorganisms, cells or mixtures thereof.

The invention relates to a microcapsule, especially for the immobilization of chemical and/or biological material, and to a method for the production thereof, which is stable in concentrated media, but is destroyed, upon diluting the medium, already under a relatively small mechanical stress, so that the encapsulated material is released. In accordance with the present invention, this material may be both a chemical substance, such as an active substance or an enzyme etc., or also a biological material, such as microorganisms, cells or mixtures thereof. Such a capsule is preferably formed of a spherical core containing the immobilized material, which may be surrounded by an envelope which encloses this core entirely.

The invention moreover relates to a microcapsule, especially for the immobilization of chemical and/or biological material, and to a method for the production thereof, which is destroyed in a deferred manner by means of an enzymatic and/or other chemical or physical process under a relatively small mechanical stress, so that the encapsulated material is released. In accordance with the present invention, this material may be both a chemical substance, such as an active substance etc., or also a biological material, such as microorganisms, cells or mixtures thereof. The capsule contains in its interior, for example, an enzyme being inactive during the storage of the capsule, but which may be activated by external factors. As a result of this activation, the enzyme breaks down one or more components of which the capsule is formed. By this, the originally stable capsule becomes mechanically instable, and the encapsulated material may be released already under a relatively small mechanical stress.

In technological practice it frequently occurs that different substances or organisms interacting with each other in a moist environment and thereby destroying each other, have to be integrated into mixtures. Normally, the components are integrated individually and in a dry form, and the mixture is stored in dry form.

If it is desired to produce similar mixtures as liquid concentrates, the possibilities are very limited. One either waives a few critical components, or one tolerates only relatively short keeping properties.

By encapsulating the critical components this disadvantage may be compensated, because it is guaranteed that these no longer interact with their surrounding medium. The applied encapsulating technique must ensure, however, that the encapsulated material is released again in case of need, so that the mixture is able to unfold its full effectiveness.

The term “encapsulation” is widespread in technical literature. In technical standards mainly those methods have so far been adopted, in which carrier particles are produced first, which are then supplied with an active substance. Frequently also so-called immersion processes are called encapsulation, e.g. as used for the production of drugs or sweets, or also spray coatings, which are frequently used in the chemical or pharmaceutical industry, whereby solids are coated with a membrane by means of immersion or spray-coating. For special applications substances are furthermore frequently encapsulated in gel particles by chemical precipitation.

Also, there are numerous protection rights or applications for protection rights the subject matter of which relates to microcapsules. For example, the unexamined laid-open patent application DE 196 44 343 A1 describes a microcapsule with a diameter of some um, which is produced in an emulsion process, in which oils or substances soluble in this oil are emulsified in a base material, e.g. alginate, from which capsules having a size of 0.5-20 um are formed in a further emulsion process, which can then be used in the food industry or the pharmaceutical industry. These globules are, however, not suited to immobilize larger solid particles or even living cells. Also, they cannot be used for the purpose according to the invention.

The U.S. Pat. No. 4,389,419 describes a similar method for encapsulating oils or oil-soluble substances. Similar to the aforementioned protection right an emulsion of the oil with a base substance (alginate) is formed in a first step. However, in this case some fillers are admixed to the alginate, and the capsules are formed by extrusion through a nozzle and precipitation in a precipitation bath, and not by an additional emulsifying step. These capsules are larger than the ones described in the first protection right. Under a higher mechanical stress the capsules bleed, similar to an oil-impregnated sponge, which likewise distinguishes them from those described in the present invention.

A class for themselves are so-called membrane capsules. F. Lim and A. Sun describe in the magazine “Science”, volume 210, pages 908-910, 1980, a capsule having a semi-permeable membrane for the immobilization of living cells, whereby the core of the capsule is surrounded by a single layer of an Ply-I-Lysin/alginate complex. With these capsules, the cells are prevented from escaping from the core of the capsule. However, it is impossible to encapsulate therein molecules having the size of an enzyme or smaller, as the membrane is permeable with respect to the same. Moreover, this membrane capsule is not suited for the use in technical processes owing to its relatively small mechanical stability.

The patent application DE 43 12 970.6 describes a membrane capsule which is also suited for the immobilization of enzymes and proteins. The core containing the immobilized material is surrounded by a multilayer envelope, with each of these layers imparting a certain property to the entire envelope. By selecting the envelope polymers in an advantageous manner the permeability of the membrane can be reduced such that the enzymes remain in the capsule, while the much smaller substrates and products can pass the membrane. These capsules are stable at very different concentrations of the surrounding medium. The membranes have a permanently adjusted defined permeability and thus prevent a release of the encapsulated material.

The patent document EP 0 782 853 B1 describes a microcapsule the envelope of which is formed of several special layers. At least one of these layers is made of a material changing its structure and thus the pore size of the envelope as a function of an internal concentration and/or other physical quantities. With this capsule, the core always remains preserved. Merely the envelope changes its permeability, which allows a partial but not a complete release of the encapsulated material.

Also in the field of detergents or cosmetic products there are a number of publications describing products with encapsulated active substances. However, all of these publications describe capsules which are either absolutely unsuitable for biological living material or which release the encapsulated substance only under a relatively high stress.

Thus, for example, aqueous washing-up liquids are known from the unexamined laid-open patent application DE 22 15 441, which comprise capsules made of the polymers Carrageenan, polyvinyl alcohol or cellulose ether. The polymers and the electrolytes are selected to ensure the stability of the capsules in the medium and the dissolution thereof when diluted. The polymers as used are not suitable for the encapsulation of living material, however.

The British patent document 1 471 406 relates to liquid aqueous detergents comprising capsules with a diameter of 0.1 to 5 mm. These capsules are to render sensitive ingredients more stable with respect to temperature, storage and transport and release the same only immediately before or during the application. The ingredients are either wholly or partially encapsulated by the capsules, with said capsules not being defined in more detail.

The German application DE 199 18 267 also describes liquid detergents comprising encapsulated ingredients. The capsules according to this invention are defined as all materials encapsulated in tenside-stable spheres available on the market. A method for producing the capsules is not described, however.

Moreover, it frequently occurs in practice that certain active substances or also microorganisms are to unfold their effectiveness in a deferred manner. This can, for example, be the case if a specific component of a mixture is to be inactive during the storage of the latter, but has to unfold its full effectiveness during the use.

An example for this would be fruit juice concentrates which, when stored as a gel, contain active substances which are to be activated only upon dilution, i.e. upon the addition of water.

With respect to baking mixtures it is frequently required that certain components have to be additionally protected during the storage. However, this protection must be inactivated at he use thereof, so that the ingredients can unfold their full effectiveness.

With chemical or biological pesticides it is often desirable that these, upon their application, unfold their effectiveness either over a longer period or only in a deferred manner.

This can be achieved with the aforementioned encapsulation of individual components. The encapsulation technique as used has to ensure, however, that the encapsulated material is released again in case of need, as to allow the substance or organism to unfold its full effectiveness. This release must be controllable by external factors such as moisture, dilution or the addition of a specific substance.

On the basis of this it is the object of the invention to provide a capsule which may contain both lifeless substances as well as living organisms. At the same time, its mechanical stability can be adjusted such that it is usable in technical processes, whereby it is stable in concentrated media, and is destroyed upon the change or dilution of the medium under a relatively small mechanical stress, so that the encapsulated material is released. The capsule is to be usable in a plurality of media, is not to be influenced by bleeding and, at the same, the drying thereof is to be possible without suffering losses of the function thereof.

In accordance with the invention the object is achieved according to a first aspect in that, in a first step, a capsule is produced in a liquid phase, which fully encloses the material to be encapsulated. In a second step, the capsule is dried. Subsequently, it is stored in the concentrate (e.g. a gel) for several hours. Upon the dilution of the capsule-concentrate-suspension high tensions are built up in the capsule to such an extent that a small mechanical stress entails its destruction and the release of the encapsulated material.

Hence, the central idea of the invention according to the first aspect resides in that the matrix, of which the capsule is formed, is initially dried and subsequently impregnated with a concentrated medium, so that, upon the dilution of the surrounding medium, the capsule is destroyed as a result of the tension built up in the interior thereof. The material encapsulated in the capsule matrix is thereby released. To prevent the material disposed in the capsule from bleeding while the capsule is stored, the capsule may be surrounded by an envelope membrane.

Upon suitably selecting the materials and the parameters of the production method, a number of various materials may be encapsulated in such a capsule, such as:
water-soluble or water-insoluble materials
greases, oils, emulsions or suspensions
solids
living or dead cells
living or dead microorganisms
mixtures from one or more of the aforementioned classes

This capsule is built up as follows: The capsule core is made of a base material, from which a matrix is formed, into which the material to be immobilized is embedded. This base substance must be a substance of which drops can be produced, from which preferably spherical porous particles may be formed by means of a precipitation through the influence of ions or a temperature gradient. Such substances can, for example, be Na-alginate, but also agarose or sephadex etc.

If, for example, enzymes or oils, emulsions etc., or also living cells or organisms, are to be encapsulated by the capsule, or in all other cases in which a specific mechanical stability of the capsule is to be adjusted, it is an advantage to envelope the capsule core with an additional membrane. This membrane may be made of a polyelectrolyte complex, which may be applied in several layers. Such polyelectrolyte complexes are formed by the interaction of a polyanion and polycations. For example, water-soluble cellulose derivatives such as carboxymethyl cellulose, cellulose sulfate or also pectines, alginates, but also synthetic polymers such as polyacrylic or polymethacrylic acids etc. can be used as polyanion. Above all natural materials such as chitosan, but also synthetic polymers like polyethylene imine or polydiethyldiallyl ammonium chloride are considered as polycation.

However, the aforementioned membrane can also be produced by a direct coating with different substances. This coating can either be accomplished during a possible drying of the capsules or be applied in a subsequent coating process. If the capsules are to be applied in the food processing industry or the pharmaceutical industry, this coating substance can either be, for example, shellac or another substance approved of for the respective field. In the chemical field also other film-forming compounds such as nitrocellulose derivatives or polyvinyl acetates etc. may be used for this purpose.

In some cases it may also be an advantage to produce a capsule by a combination of both aforementioned approaches. With such a capsule one would have an additional parameter to influence the storage properties and to prevent a bleeding of the encapsulated material and/or an interaction with the surrounding medium during the storage, by which its reliability is increased.

A method for producing an inventive microcapsule according to the first aspect is as follows:

In a first step, the material to be encapsulated is stirred into a 1-2% base material solution, e.g. Na-alginate. Next, a filler such as silica sand or silica is added, so that the mixture subsequently has a dry content of, for example, approximately 20-40%. This mixture is then instilled into a precipitation bath. This instillation may be accomplished with any commercially available system providing for uniform drop sizes. Best results were obtained with so-called two-media nozzles. These are nozzles where the drop separation at the capillaries, through which the mixture is pressed, is effected by a concentrated air stream.

The precipitation bath may be a 1-2% saline solution containing one or more multivalent metal ions, e.g. Ca⁺⁺, Ba⁺⁺ etc. If a diluted solution of a polycation is added to the precipitation bath, e.g. polyethylene imine, chitosan etc., a thin membrane is formed simultaneously with the precipitation, which prevents the encapsulated material from bleeding out of the capsule. By repeatedly flowing differently charged polyelectrolyte solutions round this capsule a membrane can be built up, which provides the capsule with a mechanical stability in correspondence with the application thereof. With this process it is an advantage that this flowing-round is accomplished with a fluidized bed. To this end, the capsules are flown round by the coating solutions in a suitable vessel at a speed high enough to not only whirl the globules, but to also keep them suspended.

Subsequently, the globules are washed and dried. The drying may be accomplished with commercially available driers, whereby the best results were achieved with fluidized-bed or vibration charge driers. The degree of moisture after the drying should not be higher than 3-7%.

After the drying, the capsule is stored in the concentrated medium, e.g. a gel, for several hours. Best results were achieved with a storage of more than 24 hours. If this concentrated medium (gel) is now diluted by the factor 5, 10 or higher, the globules are destroyed and release the encapsulated material.

According to a second aspect the object is achieved in that, in a first step, a capsule is produced in a liquid phase, which fully encloses the material to be encapsulated. In a second step, the capsule is dried. The matrix substance of the capsule or the envelope contains at least one material changeable either enzymatically or by other physical and/or chemical processes, so that the mechanical stability of the entire capsule is no longer provided.

This change can, for example, be caused by an enzyme, which is contained in the capsule and which is inactive in the dry capsule or under the conditions under which the capsule is stored. By moistening the capsule, or by a change of the physical/chemical parameters of the surrounding medium, the enzyme becomes active. By this, the enzyme breaks down one or more components of the capsule. The same becomes mechanically instable and releases the encapsulated material. However, this effect cannot only be achieved enzymatically, because, if the capsule is hardened in a reversible process, which can be reversed by adding special reagents, the capsule can be dissolved later, so that the encapsulated material is released.

Hence, the central idea of the invention according to the second aspect resides in selecting the matrix and/or a possible envelope, from which the capsule is made, such that it can be destroyed by a change of external physical and/or chemical parameters. This can, for example, be accomplished by the activation of an enzyme in the interior of the capsule, which breaks down the essential components of the capsule material. This destruction of the capsule may, however, also be accomplished differently. The material encapsulated in the capsule matrix is thereby released. To prevent the material disposed in the capsule from bleeding while the capsule is stored, the capsule may be surrounded by an envelope membrane.

Upon suitably selecting the materials and the parameters of the production method, a number of various materials may be encapsulated in such a capsule, such as:
water-soluble or water-insoluble materials
greases, oils, emulsions or suspensions
solids
living or dead cells
living or dead microorganisms
mixtures from one or more of the aforementioned classes

This capsule is built up as follows: The capsule core is made of a base material, from which a matrix is formed, into which the material to be immobilized is embedded. This base substance must be a substance of which drops can be produced, from which preferably spherical porous particles may be formed by means of a precipitation through the influence of ions or a temperature gradient. Such substances can, for example, be Na-alginate, but also agarose or sephadex etc.

If, for example, enzymes or oils, emulsions etc., or also living cells or organisms, are to be encapsulated by the capsule, or in all other cases in which a specific mechanical stability of the capsule is to be adjusted, it is an advantage to envelope the capsule core with an additional membrane. This membrane may be made of a polyelectrolyte complex, which may be applied in several layers. Such polyelectrolyte complexes are formed by the interaction of a polyanion and polycations. For example, water-soluble cellulose derivatives such as carboxymethyl cellulose, cellulose sulfate or also pectines, alginates, but also synthetic polymers such as polyacrylic or polymethacrylic acids etc. can be used as polyanion. Above all natural materials such as chitosan, but also synthetic polymers like polyethylene imine or polydiethyldiallyl ammonium chloride are considered as polycation.

However, the aforementioned membrane can also be produced by a direct coating with different substances. This coating can either be accomplished during a possible drying of the capsules or be applied in a subsequent coating process. If the capsules are to be applied in the food processing industry or the pharmaceutical industry, this coating substance can either be, for example, shellac or another substance approved of for the respective field. In the chemical field also other film-forming compounds such as nitrocellulose derivatives or polyvinyl acetates etc. may be used for this purpose.

In some cases it may also be an advantage to produce a capsule by a combination of both aforementioned approaches. With such a capsule one would have an additional parameter to influence the storage properties and to prevent a bleeding of the encapsulated material and/or an interaction with the surrounding medium during the storage, by which its reliability is increased.

A method for producing an inventive microcapsule according to the second aspect is as follows:

### EXAMPLE 1

### Capsule with Enzymatic Release

In a first step, the material to be encapsulated is stirred into a 1-2% base material solution, e.g. Na-alginate. This base material solution may also contain pectin in a concentration similar to the alginate. If required, a filler such as silica sand or silica may be added subsequently, so that the mixture subsequently has a dry content of, for example, approximately 20-40%. In addition, pectinase in a concentration of 10,000 U/kg mixture is added to the mixture. This mixture is then buffered to a pH of approximately 4 and is instilled into a precipitation bath. This instillation may be accomplished with any commercially available system providing for uniform drop sizes. Best results were obtained with so-called two-media nozzles. These are nozzles where the drop separation at the capillaries, through which the mixture is pressed, is effected by a concentrated air stream.

The precipitation bath may be a 1-2% saline solution containing one or more multivalent metal ions, e.g. Ca⁺⁺, Ba⁺⁺ etc. If a diluted solution of a polycation is added to the precipitation bath, e.g. polyethylene imine, chitosan etc., a thin membrane is formed simultaneously with the precipitation, which prevents the encapsulated material from bleeding out of the capsule. By repeatedly flowing differently charged polyelectrolyte solutions round this capsule a membrane can be built up, which provides the capsule with a mechanical stability in correspondence with the application thereof. To this end, diluted solutions of chitosan, polyethylene imine etc. may be used as polycation. Diluted solutions of pectines, alginate etc. may be used as polyanion. With this process it is an advantage that this flowing-round is accomplished with a fluidized bed. To this end, the capsules are flown round by the coating solutions in a suitable vessel at a speed high enough to not only whirl the globules, but to also keep them suspended.

Subsequently, the globules are washed and dried. The drying may be accomplished with commercially available driers, whereby the best results were achieved with fluidized-bed or vibration charge driers. The degree of moisture after the drying should not be higher than 3-7%.

After the drying, the capsule is stored in a dry condition and at a low temperature. If the capsule is moistened later, the enzyme is activated and breaks down the polyguluronic chain of the pectin portions and other corresponding components of the capsule (e.g. alginate) in both the core and the envelope. Thus, the capsule is destabilized to an extent that small mechanical stresses are sufficient to destroy the capsule and release the encapsulated material.

### EXAMPLE 2

### Capsule with Physical/Chemical Release

Like in example 1, here too, the material to be encapsulated is stirred into a 1-2% base material solution, e.g. Na-alginate, in a first step. However, the addition of pectin or the pectinase may be waived. Like in example 1, a filler such as silica sand or silica may be added subsequently, so that the mixture subsequently has a dry content of, for example, approximately 20-40%. This filler may also be omitted, however. This mixture is then instilled into a precipitation bath. This instillation may be accomplished with any commercially available system providing for uniform drop sizes. Best results were obtained with so-called two-media nozzles. These are nozzles where the drop separation at the capillaries, through which the mixture is pressed, is effected by a concentrated air stream.

If required, the obtained particles may be coated in accordance with the description of example 1. Although the globules are ready for use also in a wet condition, it is preferable to dry them.

The drying may be accomplished with commercially available driers, whereby the best results were achieved with fluidized-bed or vibration charge driers. The degree of moisture after the drying should not be higher than 3-7%. A medium again destroying the so obtained capsules is, for example, a 1-2% aqueous solution of Na-citrate. Alternatively, also a lye with a strongly alkaline pH-value may be applied.

If one contacts the capsules with such a medium, the reversible gelling process in the precipitation bath is reversed and the capsule is dissolved.

If the dried capsules are added, for example, to a dry baking mixture, which also contains Na-citrate, and moistens this mixture, the globules are destroyed and release the encapsulated material.

The destruction of the capsule can also be achieved if complexing agents in the medium surrounding the capsule matrix extract ions from the same and thereby destabilize it. Such complexing agents are frequently provided in detergents. If these extract the Ca⁺⁺ from a capsule matrix formed, for example, of Ca-alginate, the same will preserve its spherical shape in the gel, but, due to its small stability upon diluting the surrounding gel, it will be destroyed completely already under a small mechanical stress. Thus, the material encapsulated in the capsule is released. If such a capsule comprises an envelope membrane, the stability in the gel can be controlled with the influence of the complexing agents. With such an operative mechanism the capsule can be applied in both a moist and dried condition.

1. A microcapsule, especially for immobilizing solids, liquids, cells, microorganisms and/or mixtures from these materials for use in chemistry, food technology and/or pharmacy, comprising a preferably spherical core containing the material to be immobilized and preferably an envelope enclosing the same,
characterized in that
the capsule is stable when stored in a concentrated medium and, upon the dilution of the same, is destroyed already under a small mechanical stress,
the material to be immobilized is completely enclosed by the matrix (the capsule core),
the capsule core may be surrounded by a multilayer envelope completely enclosing the same,
the envelope contains at least one layer covalently and/or electrostatically bonded to the adjacent one underneath and/or above the same,
at least one layer of the envelope is applied to the capsule core by coating with a solid,
at least one layer of the envelope is formed on the capsule core by a membrane-forming liquid,
at least one layer of the envelope is formed by both complexing and coating.

2. A microcapsule according to claim 1,
characterized in that
the substance forming the core matrix is a substance of which drops can be produced, from which preferably spherical particles can be formed by means of a precipitation through the influence of ions or a temperature gradient.

3. A microcapsule according to claim 1,
characterized in that
the substance surrounding the material to be immobilized in the interior of the core matrix may be an oil or another liquid immiscible with the matrix substance such as a hydrocarbon, a hydrocarbon mixture.

4. A microcapsule according to claim 1,
characterized in that
the material to be immobilized forms a suspension with a second substance disposed in the interior of the capsule, which forms a phase boundary with the matrix substance.

5. A microcapsule according to claim 1,
characterized in that
the material to be immobilized forms a liquid/liquid emulsion with a second substance disposed in the interior of the capsule, which forms a phase boundary with the matrix substance.

6. A microcapsule according to claim 1,
characterized in that
the core matrix or at least one phase in the capsule core contains a solid.

7. A microcapsule according to claim 1,
characterized in that
at least one phase in the capsule core contains a liquid.

8. A microcapsule according to claim 1,
characterized in that
at least one phase in the capsule core contains cells.

9. A microcapsule according to claim 1,
characterized in that
at least one phase in the capsule core contains microorganisms.

10. A microcapsule according to claim 1,
characterized in that
the coating of the capsules is accomplished in a fluidized-bed process.

11. A microcapsule according to claim 1,
characterized in that
it can be dried without suffering significant losses of functionality.

12. A microcapsule according to claim 1,
characterized in that
the drying of the capsules is accomplished in a fluidized-bed process.

13. A microcapsule according to claim 1,
characterized in that
while its drying in a fluidized-bed process, a solid is additionally blown into the column which forms a membrane on the capsule surface.

14. A microcapsule according to claim 1,
characterized in that
all materials from which it is formed may be food-approved.

15. A microcapsule according to claim 1, during the production of which some, several or all of the following steps are performed, of which some steps may also be repeated several times:
dissolving or suspending the material to be encapsulated in a base material
producing drops of this base material suspension or solution
precipitating the drops
sealing the drops with the simultaneous precipitation by flowing round the globules in a precipitation bath which also contains an ionic polymer solution in addition to the precipitating reagent
rinsing and suspending the globules formed by the precipitation in a washing liquid
flowing round the globules with a cationic or anionic polymer solution and forming a cationic or anionic charge on the surface of the sphere
washing the globules with a washing liquid
flowing round the globules with an anionic or cationic polymer solution and
forming an ionic or cationic charge on the surface of the sphere
rinsing and suspending the globules formed by the precipitation in a washing liquid
drying the globules
placing (suspending) the dried globules in a concentrated medium such as a gel
storing the globules in this concentrated medium over a certain period.

16. A microcapsule according to claim 1,
characterized in that
the deliberate destruction of the capsule is achieved if complexing agents in the medium surrounding the capsule matrix extract ions from the same and destabilize the capsule.

17. A microcapsule, especially for immobilizing solids, liquids, cells, microorganisms and/or mixtures from these materials for use in chemistry, food technology and/or pharmacy, comprising a preferably spherical core containing the material to be immobilized and preferably an envelope enclosing the same,
characterized in that
the capsule is stable when stored and contains at least one component resulting in a destruction of the capsule and release of the encapsulated material by a later change of the physical and/or chemical parameters of the surrounding medium,
the component contained in the capsule, resulting in a destruction of the capsule and release of the encapsulated material by a later change of the physical and/or chemical parameters of the surrounding medium, is an enzyme capable of breaking down specific substances of the capsule matrix and/or the capsule membrane,
the component contained in the capsule, resulting in a destruction of the capsule and release of the encapsulated material by a later change of the physical and/or chemical parameters of the surrounding medium, is a substance that can be broken down by an enzyme contained in the capsule,
the component contained in the capsule, resulting in a destruction of the capsule and release of the encapsulated material by a later change of the physical and/or chemical parameters of the surrounding medium, is a substance that can be precipitated in a reversible process by the influence of ions,
the material to be immobilized is completely enclosed by the matrix (the capsule core),
the capsule core may be surrounded by a multilayer envelope completely enclosing the same,
wherein the envelope contains at least one layer covalently and/or electrostatically bonded to the adjacent one underneath and/or above the same,
at least one layer of the envelope is applied to the capsule core by coating with a solid,
at least one layer of the envelope is formed on the capsule core by a membrane-forming liquid, and/or
at least one layer of the envelope is formed by both complexing and coating.

18. A microcapsule according to claim 17,
characterized in that
the substance forming the core matrix is a substance of which drops can be produced, from which preferably spherical particles can be formed by means of a precipitation through the influence of ions or a temperature gradient.

19. A microcapsule according to claim 17,
characterized in that
the substance surrounding the material to be immobilized in the interior of the core matrix may be an oil or another liquid immiscible with the matrix substance such as a hydrocarbon, a hydrocarbon mixture.

20. A microcapsule according to claim 17,
characterized in that
the material to be immobilized forms a suspension with a second substance disposed in the interior of the capsule, which forms a phase boundary with the matrix substance.

21. A microcapsule according to claim 17,
characterized in that
the material to be immobilized forms a liquid/liquid emulsion with a second substance disposed in the interior of the capsule, which forms a phase boundary with the matrix substance.

22. A microcapsule according to claim 17,
characterized in that
the core matrix or at least one phase in the capsule core contains a solid.

23. A microcapsule according to claim 17,
characterized in that
at least one phase in the capsule core contains a liquid.

24. A microcapsule according to claim 17,
characterized in that
at least one phase in the capsule core contains cells.

25. A microcapsule according to claim 17,
characterized in that
at least one phase in the capsule core contains microorganisms.

26. A microcapsule according to claim 17,
characterized in that
the coating of the capsules is accomplished in a fluidized-bed process.

27. A microcapsule according to claim 17,
characterized in that
it can be dried without suffering significant losses of functionality.

28. A microcapsule according to claim 17,
characterized in that
the drying of the capsules is accomplished in a fluidized-bed process.

29. A microcapsule according to claim 17,
characterized in that
while its drying in a fluidized-bed process, a solid is additionally blown into the column which forms a membrane on the capsule surface.

30. A microcapsule according to claim 17,
characterized in that
all materials from which it is formed may be food-approved.

31. A microcapsule according to claim 17, during the production of which some, several or all of the following steps are performed, of which some steps may also be repeated several times:
dissolving or suspending the material to be encapsulated in a base material
adding an enzyme capable of breaking down specific components of this mixture and/or capsule coating
producing drops of this base material suspension or solution
precipitating the drops
sealing the drops with the simultaneous precipitation by flowing round the globules in a precipitation bath which also contains an ionic polymer solution in addition the precipitating reagent
rinsing and suspending the globules formed by the precipitation in a washing liquid
flowing round the globules with a cationic or anionic polymer solution and forming a cationic or anionic charge on the surface of the sphere
washing the globules with a washing liquid
flowing round the globules with an anionic or cationic polymer solution and forming an ionic or cationic charge on the surface of the sphere
rinsing and suspending the globules formed by the precipitation in a washing liquid
drying the globules
placing (suspending) the dried globules in a concentrated medium such as a gel
storing the globules in this concentrated medium over a certain period.
